(19) United States
(12) Patent Application Publication
(10) Pub. No.: US 2020/0383845 A1
(43) Pub. Date: Dec. 10, 2020

(54) BIODEGRADABLE SANITARY NAPKIN AND ABSORBATION PAD FOR HUMAN BODY

(71) Applicant: Byungyong KIM, Gwacheon-si Gyeonggi-do (KR)

(72) Inventor: Byungyong KIM, Gwacheon-si Gyeonggi-do (KR)

(21) Appl. No.: 16/762,927

(22) PCT Filed: Oct. 29, 2018
(86) PCT No.: PCT/KR2018/012879

§ 371 Date: May 9, 2020

(30) Foreign Application Priority Data
Nov. 10, 2017 (KR) 10-2017-0149690

(51) Int. Cl. A61F 13/53; A61F 13/49; A61F 13/472; A61F 13/84; A61L 15/40; A61L 15/26

(52) U.S. Cl. CPC A61F 13/53; A61F 13/49; A61F 13/472; A61F 13/5611; A61L 15/40; A61L 15/26; A61F 13/8405

(57) ABSTRACT

According to an embodiment of the present invention, a sanitary pad using a seaweed or a seagrass containing water-soluble fiber as a natural absorbent is provided. The seaweed includes any one of a brown seaweed, kelp, Sargassum, Algue brune, Ulva linza, Monostroma, dried laver, woodfish (Gelidium amansii), and Gloiopeltis furcata. The natural absorbent has a particle shape obtained by drying at least more than one of the seaweeds and the seagrasses, and the average diameter of the particles may range from 0.1 mm to 5 mm. The natural absorbent may have biodegradable properties. The natural absorbent may further include coffee grounds in the form of particles.

### CROSS-REFERENCES TO RELATED APPLICATION

The present application claims the benefit of Korean application number 10-2017-0149690, filed on Nov. 10, 2017, which is herein incorporated by reference in its entirety.

### BACKGROUND OF THE INVENTION

### 1. Field

The present invention relates to a hygiene product, and more particularly, to a biodegradable sanitary pad containing a natural absorbent and an absorption pad for a human body.

### 2. Description of the Related Art

The sanitary pad is a hygiene product that absorbs blood discharged when a woman is menstruated, and the size may be selected according to the amount of menstruation during menstruation, so that an adhesive surface may be attached to the underwear. The sanitary pad may be divided into a disposable sanitary pad, a cotton sanitary pad, and an internal sanitary pad (tampon, menstrual cup) according to the shape or material. In general, a disposable sanitary pad is widely and frequently used.

The disposable sanitary pad is composed of a triple structure of a cover, an absorbing body, and a waterproof layer. The cover is made of polyethylene, artificial fibers, or rayon as a portion that comes into contact with the skin, and the absorbing body may be made of pulp or super absorbent polymer (SAP) to absorb menstrual blood. In general, the super absorbent polymer is a polymer material capable of absorbing moisture corresponding to hundreds of times its own weight, and may absorb menstrual blood through the osmotic pressure principle. Finally, the waterproof layer is mainly made of a polyethylene component, and serves to prevent menstrual blood absorbed by the absorbing body from leaking out of the sanitary pad.

The super absorbent polymer used as an absorbing body of a conventional sanitary pad is manufactured in a particle form by using a petroleum compound such as acrylic acid polymer or polyvinyl alcohol as a raw material, and has a property of absorbing water of several hundred to 1,000 times its own weight. Currently, in connection with most sanitary pad and absorption pad, petrochemical absorbents are used in the form of particles made of sodium polyacrylate. However, since the part in contact with the sanitary pad is the most sensitive skin tissue in the body and is directly connected to the organs of the body, skin diseases such as itching and rash depending on the constitution, or menstrual pain or menstrual disorder caused by the growth of bacteria may be generated. Rarely, the influx of potentially carcinogenic chemicals into the body may cause serious side effects, such as infertility in women. In addition, due to its strong absorption power, it absorbs not only menstrual blood, but also beneficial moisture in the contacted human body, causing skin dryness, which may cause various female diseases.

For this reason, chlorine-free natural pulp, which is relatively safe for the human body, such as cellulose collected from 100% pulp, is used as an absorbing body. However, due to a manufacturing process in which wood pulp is interlocked and fixed using multiple layers of starch, the manufacturing cost increases, and it is difficult to produce a universal and popular product by using natural pulp rather than recycled pulp. In addition, it is difficult to consider it as environmentally friendly because the forest is damaged for pulp production.

In addition, since the sanitary pad used once is not soluble in water, it must be collected and treated separately. Therefore, a waste sanitary pad becomes a source of environment pollution.

Accordingly, the technological problem to be solved by the present invention is to provide a biodegradable and eco-friendly sanitary pad, wherein various side effects caused by the use of sanitary pad is reduced, skin protection, sterilization, and menstrual cramping are possible, absorption capacity is improved, a production cost is low, forest damage is reduced.

In addition, another technological problem to be achieved by the present invention is to provide an absorption pad for a human body having the aforementioned advantages.

### SUMMARY OF THE INVENTION

According to an embodiment of the present invention, a sanitary pad using a seaweed or a seagrass containing water-soluble fiber as a natural absorbent may be provided. The seaweed includes any one of a brown seaweed, kelp, Sargassum, Algue brune, Ulva linza, Monostroma, dried laver, woodfish (Gelidium amansii), and Gloiopeltis furcata. The natural absorbent has a particle shape obtained by drying at least more than one of the seaweeds and the seagrasses, and the average diameter of the particles may range from 0.1 mm to 5 mm. The natural absorbent may have biodegradable properties. The natural absorbent may further include coffee grounds in the form of particles.

According to another embodiment of the present invention, an absorption pad comprising a natural absorbent including seaweed or seagrass having water-soluble fiber; and an absorption cotton having a non-woven structure and accommodating the natural absorbent may be provided. The seaweed may include any one of a brown seaweed, kelp, Sargassum, Algue brune, Ulva linza, Monostroma, dried laver, woodfish (Gelidium amansii), and Gloiopeltis furcata. The sea grasses may include turtle seagrass, Zostera marina or a surf grass. The natural absorbent has a particle shape obtained by drying at least more than one of the seaweeds and the seagrasses, and the average diameter of the particles may range from 0.1 mm to 5 mm. The natural absorbent may have biodegradable properties. The absorption cotton comprises a biodegradable polyester prepolymer. The biodegradable polyester prepolymer may include polylactic acid (PLA), poly(butylenes succinate)(PBS), poly(butylene succinate-co-butylene adipate)(PBSA) and poly(butylene adipate-coterephthalate)(PBAT), PCL (Poly-Caprolactone), PGA (poly glycolic acid), PHB (poly hydroxy butyric acid), or a combination thereof.

According to an embodiment of the present invention, by using seaweed as a natural absorbent, it is possible to reduce various side effects associated with the use of sanitary pad, it is possible to protect the skin, sterilize, and relieve menstrual pain.

In addition, since the natural absorbent of the present invention has a higher content of water-soluble fiber than the conventional synthetic compound, that is, a Super Absorption Polymer (SAP), it has an excellent absorption rate of menstrual blood, and thus may improve the absorption capacity of menstrual blood.

In addition, because the material of the seaweed is abundant, it is possible to reduce the production cost of the product and reduce the damage to the forest caused by reducing the use of natural pulp.

In addition, the natural absorbent of the present invention has an eco-friendly advantage that is biodegradable in nature, thereby reducing environmental pollution due to disposal of the waste sanitary pad.

### BRIEF DESCRIPTION OF THE DRAWINGS

FIG. 1 is a perspective view of a sanitary pad according to an embodiment of the present invention.

FIG. 2 is a cross-sectional view of a sanitary pad according to an embodiment of the present invention.

FIG. 3A is a view showing a seaweed in the form of particles used as a natural absorbent of a sanitary pad according to an embodiment of the present invention.

FIG. 3 is a view showing a seaweed in the form of particles used as a natural absorbent sanitary pad according to an embodiment of the present invention

FIG. 3B to FIG. 3C are diagrams showing a pulp absorbent and a super absorbent polymer used as absorbents for sanitary pad which have generally used until now.

FIG. 3D is a view showing coffee grounds in the form of particles used as a natural absorbent of a sanitary pad according to another embodiment of the present invention.

FIG. 3E is a view showing a mixture of seaweed and coffee grounds in the form of particles used as a natural absorbent of a sanitary pad according to another embodiment of the present invention.

### DETAILED DESCRIPTION OF THE INVENTION

Hereinafter, preferred embodiments of the present invention will be described in detail with reference to the accompanying drawings.

The embodiments of the present invention are provided to more completely describe the present invention to those having a common knowledge in the related art, and the following examples may be modified in various other forms, and the scope of the present invention is not limited to the following embodiments. Rather, these embodiments are provided to describe the present invention more faithfully and completely, and to fully convey the spirit of the present invention to those skilled in the art.

In addition, in the following drawings, a thickness or a size of each layer is exaggerated for convenience and clarity of description, and the same reference numerals in the drawings refer to the same elements. As used herein, the term, “and/or” includes any and all combinations of one or more of the listed items. The terminology used herein is used to describe a specific embodiment and is not intended to limit the present invention. As used herein, singular forms may include plural forms unless the context clearly indicates otherwise.

Also, as used herein, the term, “comprise” and/or “comprising” specifies the mentioned shapes, numbers, steps, operations, members, elements and/or the presence of these groups, and does not exclude the presence or addition of one or more other shapes, numbers, steps, operations, members, elements and/or existence or addition of these groups.

Although the terms, first, second, etc. are used herein to describe various members, parts, regions, layers and/or parts, it is obvious that these members, parts, regions, layers and/or parts are not defined by these terms. These terms are only used to distinguish one member, component, region, layer or part from another region, layer or part. Accordingly, the first member, component, region, layer or part described below may refer to the second member, component, region, layer or part without departing from the teachings of the present invention.

Hereinafter, embodiments of the present invention will be described with reference to the drawings schematically showing ideal embodiments of the present invention. In the drawings, for example, the size and shape of members may be exaggerated for convenience and clarity of description, and in actual implementation, variations of the illustrated shape may be expected. Accordingly, the embodiments of the present invention should not be construed as limited to the specific shapes of the members or regions shown herein. Hereinafter, various embodiments of the present invention will be described with reference to the drawings.

The present invention relates to an absorption pad used directly or indirectly in a human body such as a sanitary pad and a diaper using a natural absorbent and a method for manufacturing the same.

In the present invention, the various kinds of pad used in the human body such as disposable sanitary pad, diapers, and defecation pad differ only in terms of a shape, a use range, size, etc., and the general properties, product configuration and manufacturing methods are similar, and the present invention is a technology related to absorbents used in the various kinds of pad. Therefore, the most commonly used sanitary pad is suggested as a representative example to facilitate convenience and understanding of the description, and will be hereinafter collectively described

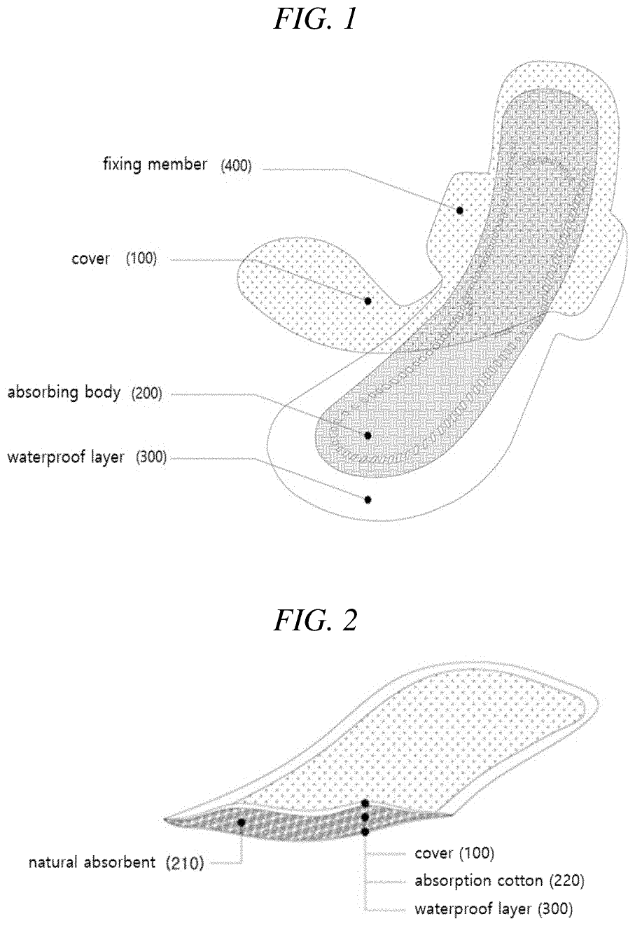

FIG. 1 is a perspective view of a sanitary pad according to an embodiment of the present invention, and FIG. 2 is a cross-sectional view of a sanitary pad according to an embodiment of the present invention.

Referring to FIG. 1, the sanitary pad 10 may include a cover 100 that comes into contact with the skin (not shown), an absorbing body 200 composed of an absorption cotton 220 absorbing menstrual blood which has permeated through the cover 100 and a natural absorbent 210, and a waterproof layer 300 that prevents leakage or staining of menstrual blood during activity. The absorbing body 200 may be disposed on the waterproof layer 300, and the cover 100 may be disposed on the absorbing body 200. In one embodiment, a fixing member 400 protruding to both sides of the cover 100 of the sanitary pad 10 and the waterproof layer 300, and fixing the sanitary pad 10 to the underwear may be further included.

The fixing member 400 may fix the sanitary pad 10 to the underwear to increase activity. In the embodiment of the present invention, the length and shape of the sanitary pad 10 is only one example, and the present invention is not limited thereto. That is, the present invention may be applied to various types of sanitary pad, and the absorption pad used in the human body. The natural absorbent 210 may be disposed as a form of a particle in the absorption cotton 003 of the form of a nonwoven fabric. Specifically, the natural absorbent 210 of a form of a particle may be trapped in the pores in the absorption cotton 003 of the form of a nonwoven fabric.

In one embodiment of the present invention, the natural absorbent 210 may include sea algae or sea grasses rich in water-soluble fiber. The algae (see algae) may be at least one of green algae, brown algae, and red algae. In these seaweeds, the green light may disappear at a shallow place and the red light may penetrate into a deep place, so that the green algae may be found in relatively shallow and bright areas, and the brown algae and the red algae in turn may be found in a deep and dark place, which is gradually getting deeper and darker. The green algae may include a sea lettuce (Ulva lactuca), Ulva linza or Monostroma. The brown algae may include seaweed, kelp, Sargassumcapsicum or Algue brune, and the red algae may include dried laver, woodfish (Gelidium amansii), and Gloiopeltis furcata. The sea grasses may include turtle seagrass, Zostera marina or a surf grass. However, the natural absorbent 210 of the present invention is not limited to these. For example, salt marsh plants or mangroves rich in water-soluble fiber, or mangroves can be used alone or in combination with them.

As described above, instead of the synthetic compound super absorbent resin(SAP) used as a conventional absorbing body, a natural absorbent of the present invention is prepared by using the substances which may be easily obtained in nature while containing various ingredients and effects beneficial to the human body, such as minerals and vitamins. Therefore, it is expected not only to improve various side effects according to the use of the sanitary pad, but also it is expected to have beneficial effects such as skin protection, sterilization, pain relief, and soothing effect. In addition, it has better absorption performance than the absorbent using natural pulp, and may be applied to existing sanitary pad manufacturing methods without a separate manufacturing process, thereby reducing the production cost as compared to natural pulp products and thereby popularizing it. Moreover, when compared to natural pulp absorbing bodies, it is possible to prevent deforestation and has an eco-friendly advantage that is biodegradable in nature.

In one embodiment, the natural absorbent 210 constituting the absorbing body 200 may be a seaweed in the form of particles among the seaweeds rich in water-soluble fiber that absorbs moisture well. The seaweed in the form of particles may be formed by finely drying the dried seaweed. In order to prepare the dried seaweed in particle form, various manufacturing methods may be applied.

Seaweed in a completely dry state contains water-soluble fiber that may absorb moisture up to 8 times the dry weight, which is the highest level among plants in nature. Therefore, 1 gram of a natural absorbent made from seaweed may absorb approximately 8 ml of menstrual blood. Generally, In the case of a typical woman, a menstruation bleeding from 50 ml to 100 ml occurs over 3 to 6 days during one menstruation once, and is different for each individual. However, usually 2 to 5 sanitary pad 10 per day may be used depending on the amount of menstrual bleeding of 5 to 15 ml per day. Therefore, when the amount of the natural absorbent 210 of the sanitary pad 10 of the present invention includes 1 g to 5 g by weight of the natural absorbent 210, the menstrual blood absorption weight is about 8 g to 40 g, which sufficiently absorbs the average daily menstrual volume. However, the content of the absorbent in the present invention is not limited to this and may be variously adjusted according to the size and characteristics of the sanitary pad 10.

In one embodiment, according to a method for pulverizing natural absorbent 210 into granules (eg., particles), and a method wherein the particle form may be manufactured by various conventional methods such as general crushing, cutting, and grinder, a dry seaweed may be prepared. But, preferably, in the present invention, dry seaweed may be crushed as the form of particles by using a method such as a mechanical roll mill operation used for grinding red pepper powder or rice. According to the present invention, by crushing the natural absorbent 210 by mechanical pressure, it softens the fibrous tissue of seaweed dried and hardened, and the contact surface area becomes large, thereby making it easier to realize menstrual blood absorption as compared to preparing particles by a cutting process. The natural absorbent 210 crushed by the above-described manufacturing method may be made into a normal size that is easy to manufacture a sanitary pad, but when the particle size is large, a foreign body feeling may be felt, and if the particle size is too small, the phenomena that the particles are collected inside the absorption cotton 003, or the particles may leak out of the cover 100 of the sanitary pad 10 may be observed. Therefore, the size of the natural absorbent 210 is not too large or small after considering the materials and characteristics of the sanitary pad 10 and the absorption cotton 220. Preferably, the diameter of the particles ranges from 0.1 mm to 5 mm and more preferably from 0.3 mm to 2 mm.

In an embodiment of the present invention, as a result of the blinding testing 20 adult women, there is an opinion that the natural absorbent 210 produced by seaweed has a fishy peculiar smell of seaweed, but this is limited to a case when smelling closely it. But, it was observed that this disadvantage was not so common when it was assumed that the normal use mode was used while being attached to the underwear. However, during menstruation, women become psychologically sensitive, and as time passes from the body and menstrual blood to the body, there are cases where the stink smell may be generated from menstrual blood and vaginal secretions. This clearly indicates the reason why women who use sanitary pad are psychologically uncomfortable in their use. In order to solve this, some sanitary pad products include herbal medicines of mugwort or compounds that cause fragrance, but this is also a factor that causes other unpleasant odors along with the stink smell of menstrual blood. Therefore, it is not desirable because there are women who dislike the scent of herbal medicine depending on their taste. Therefore, in the embodiment of the present invention, the natural absorbent made of seaweed having a high absorption rate because of high water-soluble fiber is used, and the dried coffee grounds (or waste coffee powder) after use, which is rich in insoluble fiber and has fine pores on the surface, and thus has high adsorption power against the unpleasant odors and foreign substances is also partially included to allow absorption of menstrual blood and fundamental absorption of various odors into coffee grounds. Coffee grounds, which are extracted and discarded, have the advantage that they can be used in the product without a separate process other than a dry sterilization process, and the vegetable oil contained in the coffee grounds protects the skin along with the ingredients of seaweed, and when its caffeine components are absorbed by the body, it is well known that it helps relieve blood circulation and pain in menstrual cramps, so its efficacy may be highly expected.

In addition, the coffee grounds have excellent adsorption capacity to be used as a deodorant fragrance. However, in the case of coffee grounds, it has the property of instantly absorbing moisture as much as its own weight through fine pores, but it is made of insoluble fiber and does not solidify or gel. Thus, it can not absorb moisture beyond the maximum magnetic weight and when there are external pressures, moisture tends to come out easily. Therefore, it is difficult to use it as a natural absorbent absorbing menstrual blood as in the present invention. Therefore, it is preferable to add a predetermined amount as a natural adsorbent 210 and an absorbent in consideration of deodorizing performance and diffusion of coffee flavor rather than using a large amount with a natural absorbent

The method of adding the absorbent in the general sanitary pad preparation is not to adhere or fix the absorption cotton 220, but to distribute it evenly inside the fine and homogeneous absorption cotton 220. Also, in the present invention, the method of adding the absorbent is the same as that of the general sanitary pad.

The present invention is to develop a sanitary pad having a beneficial effect on the environment without being harmful while using the natural absorbent 210. The absorption cotton 300 containing the natural absorbent 210 used in the present invention includes a biodegradable polyester prepolymer. The biodegradable polyester prepolymer is polylactic acid (PLA), poly(butylenes succinate)(PBS), poly(butylene succinate-co-butylene adipate)(PBSA) and poly(butylene adipate-coterephthalate)(PBAT), PCL (Poly-Caprolactone), PGA (poly glycolic acid), PHB (poly hydroxy butyric acid), or a combination thereof. The present invention is not limited to these and may be made from any component that is harmless to the human body. Preferably, it is preferable to use natural vegetable cotton fuzz of cotton or pulp, and more preferably, it is made of natural material of corn chitosan starch such as PLA (Poly-Lactic Acid), and as a result, it is preferable to biodegrade in nature without significantly damaging the forest.

In addition, in one embodiment, when the natural material is applied to the cover 100 and the waterproof layer 300 as well as the absorption cotton 220, a sanitary pad that is 100% biodegradable in nature with the natural absorbent 210 may be provided. Natural absorbent 210 made from seaweed has a higher absorption rate and absorption speed as compared with other sanitary pad using absorbents made from other natural plants or natural pulp containing water-soluble fiber. This is because there are many water-soluble fibers such as alginic acid in seaweed, but this is also based on the reason that mannitol (eg, D-mannitol) that induces an osmotic reaction has C6H14O6, molecular weight 182.2, melting point 168° C., non-hygroscopic needle crystal, and solubility of 18.2 g. In connection with some brown algae such as kelp and seaweed, mannitol, for example, 15% to 25%, preferably around 20% may be contained, and mannitol, for example, 10% to 20%, preferably up to 14% may be contained with trehalose and arabitol existed in mushrooms, for example, 10% to 20%, preferably up to 14%. The mannitol is also found in carrots and onions. The mannitol can also be produced from glucose and sucrose by yeast and fungi. It can be produced simultaneously with sorbitol by a contact reduction process of fructose and invert sugar. It can also be obtained by thermal ethanol extraction from kelp. Although it has a sweet taste and shows the general properties of sugar alcohol, its use in food is not known except that it is used as an anti-sticking agent for gum and syrup, and mannitol extracted from seaweed and kelp can be used in medicines such as osmotic diuretics.

### Embodiment

In the present invention, a synthetic compound superabsorbent polymer (SAP), which is an absorbent of a conventional sanitary pad, is not used. Instead, the present invention employs a kind of seaweed such as seaweed and kelp, which is abundant around the surroundings in the natural state, has a high water-soluble fiber content, has excellent moisture absorption due to the above fiber, and is rich in vitamins and minerals that are beneficial for bleeding due to women's skin and menstruation. The sanitary pad is prepared by attaching the natural absorbent prepared using the seaweed. In addition, according to the present invention, a sanitary pad made of a natural absorbent absorbs moisture from menstrual blood quickly and becomes gel, and at the same time, a small amount of iron-like components in the blood and a natural coagulation reaction occur, resulting in leakage or staining of menstrual blood.

Table 1 is a comparison table of the performance of natural absorbents made from seaweed used in the sanitary pad of the present invention. As a comparative example, an absorbent was prepared using a pulp absorbent and a super absorbent polymer

TABLE 1

| | | | initial | initial | | | | existence |
|---|---|---|---|---|---|---|---|---|
| | | absorption | absorption | absorption | gel | | | of |
| Absorbent | weight | amount | time | time | (coagulation) | staining | leaking | unpleasant |
| Sample | (g) | (ml) | (second) | (second) | reaction | reaction | reaction | odor |
| Natural | 4 | 12 | 0.3 | 3 | Existence | Nothing | Nothing | slightly |
| absorbent(sea- | | | | | | | | |
| weed 100%) | | | | | | | | |
| Pulp | 4 | 12 | 0.5 | 6 | Non- | slightly | slightly | Non- |
| absorbent(pulp | | | | | existence | | | existence |
| 100%) | | | | | | | | |
| Super absorbent | 4 | 12 | 0.1 | 4 | Existence | Nothing | Nothing | Non- |
| resin | | | | | | | | existence |
| (resin 100%) | | | | | | | | |
| Coffee | 4 | 12 | 5 | 10 | Non- | Plenty | Plenty | Non- |
| grounds(coffee | | | | | existence | | | existence |
| 100%) | | | | | | | | |
| Natural | 4 | 12 | 0.5 | 4 | Existence | Non- | Non- | Non- |
| absorbent + Coffee | | | | | | existence | existence | existence |
| grounds | | | | | | | | |

Referring to Table 1, in the present invention, the natural absorbent produced by seaweed has the fastest initial absorption rate and the final absorption rate, and the phenomenon of oozing or leaking is the same level as the currently used super absorbent resin, and It was found to be superior to natural pulp products that were known to be harmless. However, individuals can feel the unpleasant smell of sea odor, but when using natural absorbent and coffee grounds as an adsorbent, it is observed that there is no unpleasant odor and rather a coffee smell is felt. Further, it turned out that absorption power, staining or leaking reaction was also excellent. The initial absorption time was the best since the super absorbent resin reacted as soon as it touched the moisture, but it did not show a significant difference from the natural absorbent.

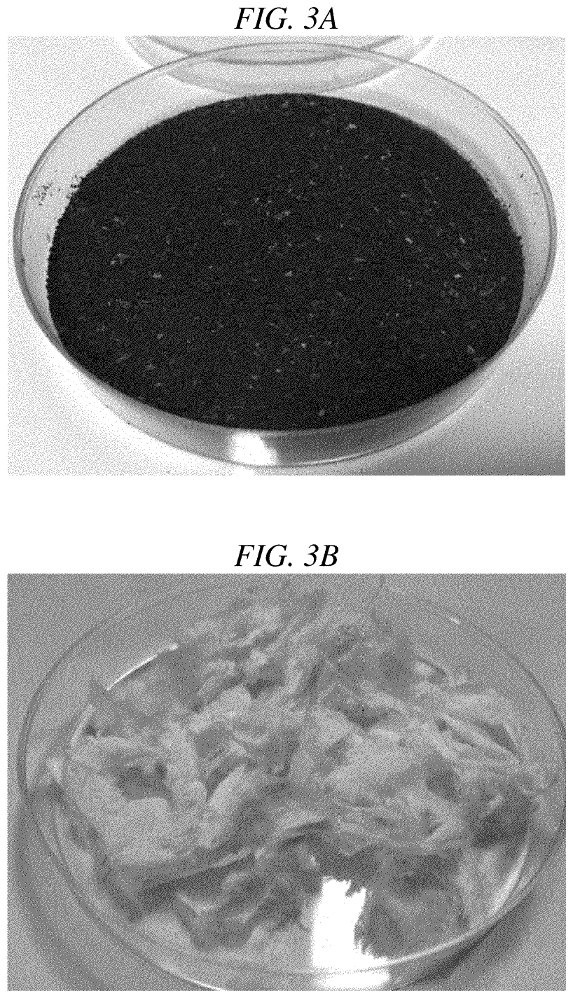

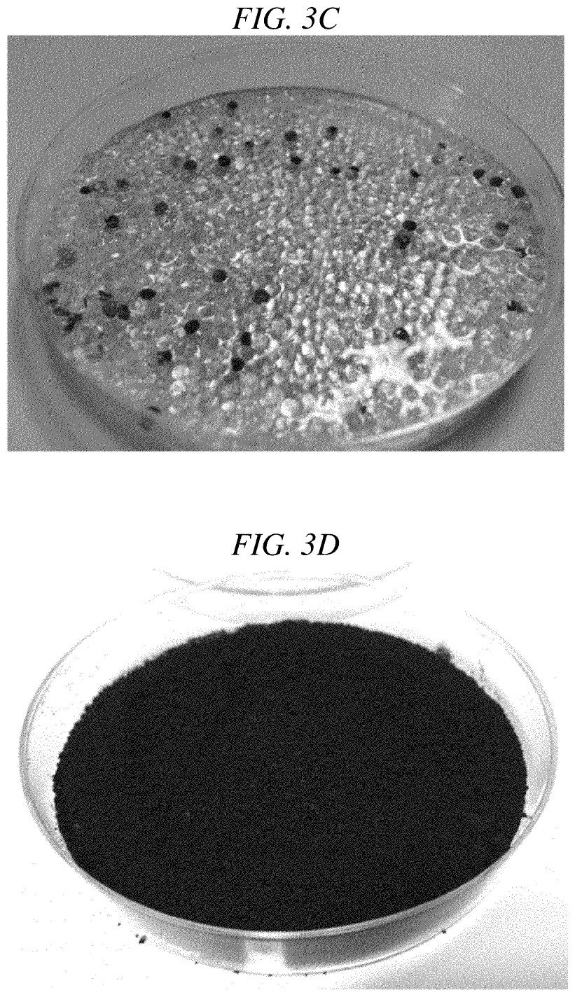

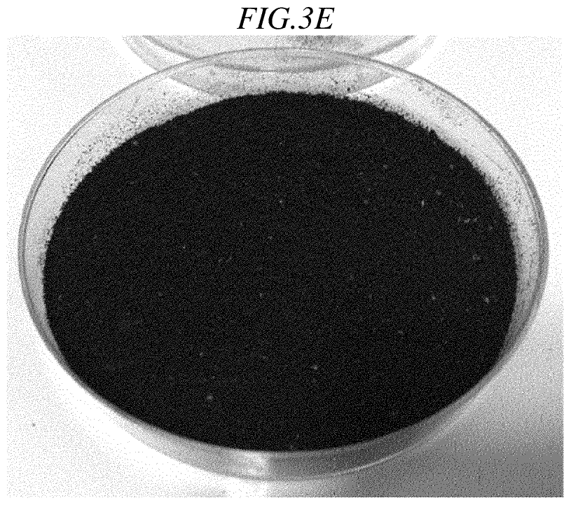

FIG. 3A is a seaweed in the form of particles used as a natural absorbent, FIG. 3B to FIG. 3C are a pulp absorbent and a super absorbent polymer used as a conventional absorbent, respectively. FIG. 3D It is a coffee ground in the form of particles used as a natural absorbent, and FIG. 3E is a mixture of seaweed and coffee grounds in the form of particles used as a natural absorbent.

In one embodiment of the present invention, the specific ingredients contained in the seaweed which is the main raw material of the natural absorbent used in the sanitary pad are as described below as a non-limiting example, and have various beneficial effects on the human body.

First of all, alginic acid contained in some seaweeds, such as seaweed, is a good water-soluble fiber and accounts for 20% to 30% of a seaweed component such as kelp. In addition, seaweed and kelp contain a lot of carbohydrate produced by carbon assimilation and alginate component, which is a component of the cell wall, as well as many pigments, such as xanthophylls and chlorophyll. In addition, it is considered as a representative health food because it is rich in amino acids such as iodine, vitamin B2, and glutamic acid.

In addition, the seagrass such as kelp are rich in carotene, vitamin A, vitamin B2, amino acids, sugars, fiber, minerals, and iodine as well as sticky alginic acid. In addition, since alginic acid itself is in a fibrous form, it helps to discharge other waste products from the body, and especially has the effect of reacting with radioactive substances such as strontium 90, and discharging the waste product. Further, there is an effect that the aforementioned ‘sodium alginate’ also has the effect of collectively discharging cadmium, barium, copper and heavy metals of manganese contained in a body.

The specific efficacy of alginic acid and seaweed, which are representative components of a seaweed verified in various conventional experiments, is as follows.

1) Picosterol, which contains 21 mg out of 100 g, is a potent carcinogen.

2) Prevents high blood pressure and lowers cholesterol in the blood.

3) Suppresses vascular disease and protects the stomach to suppress stomach cancer.

4) Anti-cancer action against lung cancer.

5) Kill 0157.

6) Acidic polysaccharides unique to seaweed acts to enhance cancer immunity.

7) There are excellent effects in anti-ulcer action, and duodenal ulcer.

8) An effect to reduce the harm of tobacco nicotine.

9) Smoothes metabolism, prevents aging, and is good for skin beauty.

10) Reduces cholesterol and shortens blood vessels.

11) Reduces blood sugar in diabetic patients.

12) Best food to solve obesity problem

13) They are very good for vascular disease, such as suppressing and improving cholesterol, suppressing blood clotting, and atherosclerosis of heart disease.

14) Relieves constipation.

15) A strong alkaline food.

Among the above-mentioned effects of seaweed, the merits can be enumerated that smooth metabolism is realized through improved blood flow, very excellent effect for skin beauty is obtained, a bacterial growth is suppressed, heavy metals and waste products in the body are absorbed, and it is very useful for anti-cancer and contraction of the uterus. Therefore, due to the characteristics of the sanitary pad used by women, an excellent pharmacological effect may be imparted to the sanitary pad. In addition, much amounts of calcium, iron, and zinc components contained in the seaweed may also helpful for curing anemia generated due to menstruation during menstruation.

As described above, in the present invention, instead of a synthetic compound of an absorbing body known to be harmful to the human body in manufacturing a sanitary pad having a structure of a cover, an absorbing body, and a waterproof layer, the seaweeds which contain the most much amount of water-soluble fiber, which is commonly obtained in the surroundings and is excellent in absorbing moisture. Therefore, the manufacturing cost may be lowered. In particular, a sanitary pad may be prepared using a natural absorbent prepared by drying seaweed containing a lot of beneficial ingredients for the skin. The present invention is not limited to this, and the seaweed similar to seaweed may be used, but the seaweed is more preferable when considering economic value and processability.

In terms of effects, as no synthetic compounds are used, there are no side effects, and various vitamins and minerals such as alginic acid, mannitol, vitamin C, vitamin A, vitamin B1, iron, calcium, and zinc, which make up mucus among natural ingredients contained in the seaweed are beneficial and useful for protecting and reproducing women's skin, promoting blood circulation, and relieving menstrual cramps, and the effects are known through various experiments and medical tests. In addition, when seaweed is dried and processed into a natural absorbent, it contains natural sodium components contained in salt as well as various minerals and vitamins, which also has the effect of suppressing the growth of bad bacteria that may occur due to physiological blood coming out of the body. In addition, when manufacturing sanitary pad by using seaweeds such as seaweed and kelp, which are commonly available in the surroundings, they have advantages such as low cost, excellent absorption, and there is a merit that mass production may be easily realized when compared to products using natural pulp absorbents.

In addition, in the case of a sanitary pad using a conventional synthetic compound, that is, a super absorbent polymer (SAP), the super absorbent polymer absorbs menstrual blood and is hardened, resulting in a foreign body sensation, which in turn leads to uncomfortable wearing of the sanitary pad. However, the sanitary pad using a natural absorbent containing the seaweed as the main component of the present invention interacts with the components such as alginic acid and mannitol, which are fibers that make up carbohydrates of seaweed when absorbing menstrual blood and other moisture. It has the advantage of not being uncomfortable when using a sanitary pad because it is gelled in a soft form such as a sponge.

According to a further embodiment of the present invention, it is also possible to enhance the effect of removing odors that may be caused by physiological or vaginal secretions by partially containing coffee grounds as a natural absorbent together with a natural absorbent made from seaweed.

However, the sanitary pad 10 according to the present invention basically removes harmful components and provides various beneficial effects on the skin of a woman according to the use of the sanitary pad. This means that sanitary pad, which are safer and can help women's health, can be mass-produced, and it is understood that the value of industrial use is very high. Furthermore, until now, the seaweed and some of the seagrass, which have been used only as a raw material for extracting certain natural ingredients such as mannitol as well as the directly ingested food and mannitol, may be applied to various pad products used as absorbents, so it may be expected to create new added value for seaweed. In addition, the seaweed is grown not only to the certain regions, but is distributed all over the world and is readily available to anyone, and is a very inexpensive raw material as compared to chemical absorbents or pulp if mass production is realized by utilizing these advantages, it may lower the consumer price of sanitary pad, and safe sanitary pad may be provided to the economically disadvantaged and the young girls at lower prices. Thus, it is considered that it has the tremendously social and industrial values.

In addition, the adsorbent utilizing coffee grounds added in small amounts has an industrial value to reduce disposal costs occurred upon disposal, and social public costs incurred by recycling waste coffee waste.

It will be apparent to those who have the common knowledge in the related art to which the present invention belongs to, that the present invention described above is not limited to the above-described embodiments and the accompanying drawings, and various substitutions, modifications, and changes are possible within the scope of the technological spirit of the present invention.

What is claimed is:

1. A sanitary pad using seaweed or seagrass containing water-soluble fiber as a natural absorbent.

2. The sanitary pad of the claim 1, wherein the seaweed includes any one of seaweed, kelp, Sargassum, Algue brune, Ulva linza, Monostroma, dried laver, woodfish (Gelidium amansii), or Gloiopeltis furcata, and the seagrass includes any one of Thalassia, Zostera marina or a surf grass (Phyllospadix).

3. The sanitary pad of the claim 1, wherein the natural absorbent has a particle shape obtained by drying at least more than one of the seaweed and the seagrass, and an average diameter of the particle ranges from 0.1 mm to 5 mm.

4. The sanitary pad of the claim 1, wherein the natural absorbent has a biodegradable property.

5. The sanitary pad of the claim 1, wherein the natural absorbent further includes coffee grounds in a form of a particle.

6. An absorption pad comprising a natural absorbent including seaweed or seagrass having water-soluble fiber; and an absorption cotton having a non-woven structure and accommodating the natural absorbent.

7. The absorption pad of the claim 6, wherein the seaweed includes any one of a brown seaweed, kelp, Sargassum, Algue brune, Ulva linza, Monostroma, dried laver, woodfish (Gelidium amansii), or Gloiopeltis furcata, and the seagrass includes any one of Thalassia, Zostera marina or a surf grass (Phyllospadix).

8. The absorption pad of the claim 6, wherein the natural absorbent has a particle shape obtained by drying at least more than one of the seaweed and the seagrass, and an average diameter of the particle ranges from 0.1 mm to 5 mm.

9. The absorption pad of the claim 6, wherein the absorption cotton comprises a biodegradable polyester prepolymer, and The biodegradable polyester prepolymer may include polylactic acid (PLA), poly(butylenes succinate)(PBS), poly(butylene succinate-co-butylene adipate)(PBSA) and poly(butylene adipate-coterephthalate)(PBAT), PCL (Poly-Caprolactone), PGA (poly glycolic acid), PHB (poly hydroxy butyric acid), or a combination thereof.
